(19) United States
(12) Patent Application Publication
(10) Pub. No.: US 2012/0112100 A1
(43) Pub. Date: May 10, 2012

(54) Bactericidal Lighting Device

(75) Inventor: Tien-Jen LO, Kaohsiung City (TW)

(73) Assignee: SINO MIND GROUP LIMITED, APIA (WS)

(21) Appl. No.: 12/942,670

(22) Filed: Nov. 9, 2010

(51) Int. Cl. G21G 5/00

(52) U.S. Cl. 250/492.1

(57) ABSTRACT

A bactericidal lighting device includes a bactericidal lighting bar and a charging base for charging the bactericidal lighting bar. The bactericidal lighting bar includes a housing, a bactericidal lamp unit for generating ultraviolet light, and a control unit. The control unit includes a control circuit operable to activate and deactivate the bactericidal lamp unit, an activate key to control activation of the control circuit for activating the bactericidal lamp unit, a safety switch to deactivate the bactericidal lamp unit when the housing is turned beyond a predetermined angle relative to a normal position, and an override key to selectively override the safety switch such that the bactericidal lamp unit is able to remain activated even when the housing is turned beyond the predetermined angle.

### BACKGROUND OF THE INVENTION

1. Field of the Invention

The invention relates to a bactericidal device, more particularly to a bactericidal lighting device capable of generating ultraviolet light.

2. Description of the Related Art

As shown in FIG. 1, Taiwanese Utility Model No. M284393 discloses a portable ultraviolet bactericidal lighting device 1. The bactericidal lighting device 1 is convenient to carry and is operable to generate ultraviolet light for bactericidal purposes. When in use, however, the eyes of a user are exposed to the risk of injury if the lighting device 1 is accidentally turned toward the face of the user while an ultraviolet lamp 11 of the lighting device 1 generates ultraviolet light. To overcome this drawback, a similar lighting device that is further provided with a safety switch has been proposed. The safety switch interrupts power when the bactericidal lighting device is turned beyond a predetermined angle relative to a normal position so as to minimize the risk of injury to the user's eyes. However, use of the safety switch will limit applications of the bactericidal lighting device.

Moreover, although the bactericidal lighting device 1 may use batteries or an external power supply as a power source, the batteries require frequent replacement, whereas the external power supply requires a power cord, the length of which restricts a serviceable range of the lighting device 1.

### SUMMARY OF THE INVENTION

Therefore, the object of the present invention is to provide a rechargeable bactericidal lighting device that is convenient to use.

Accordingly, a bactericidal lighting device of the present invention includes a bactericidal lighting bar and a charging base. The bactericidal lighting bar includes a housing having one side formed with an opening, a bactericidal lamp unit mounted in the housing and operable to generate ultraviolet light that exits the housing via the opening, a control unit connected electrically to the bactericidal lamp unit, and a power supply. The control unit includes a control circuit disposed in the housing and operable to activate and deactivate the bactericidal lamp unit, an activate key accessible from an exterior of the housing, coupled to the control circuit, and operable to control activation of the control circuit for activating the bactericidal lamp unit, a safety switch coupled to the control circuit and operable to deactivate the control circuit for deactivating the bactericidal lamp unit when the housing is turned beyond a predetermined angle relative to a normal position, and an override key accessible from the exterior of the housing, coupled to the control circuit, and operable to selectively override the safety switch such that the control circuit and the bactericidal lamp unit are able to remain activated when the housing is turned beyond the predetermined angle relative to the normal position. The power supply includes a rechargeable battery mounted in the housing and operable to supply power to the control unit and the bactericidal lamp unit, and a set of charging contacts connected electrically to the rechargeable battery and accessible from the exterior of the housing. The charging base includes a base body for holding removably the housing of the bactericidal lighting bar, a power converter disposed in the base body and operable to convert input power into battery charging power, and a set of conductive terminals accessible from an exterior of the base body, coupled to the power converter, and disposed to establish electrical connection with the charging contacts when the housing is held by the base body to permit supply of the battery charging power to the rechargeable battery.

By virtue of the designs of the bactericidal lighting bar and the charging base, the bactericidal lighting bar may be conveniently recharged, and the bactericidal lighting bar may be used for long-duration localized bactericidal operations while it is held on the charging base. The bactericidal lighting bar is also available for use in a 180° service range mode or a 360° service range mode.

### BRIEF DESCRIPTION OF THE DRAWINGS

Other features and advantages of the present invention will become apparent in the following detailed description of the preferred embodiment with reference to the accompanying drawings, of which:

FIG. 1 is a perspective view of a conventional bactericidal lighting device;

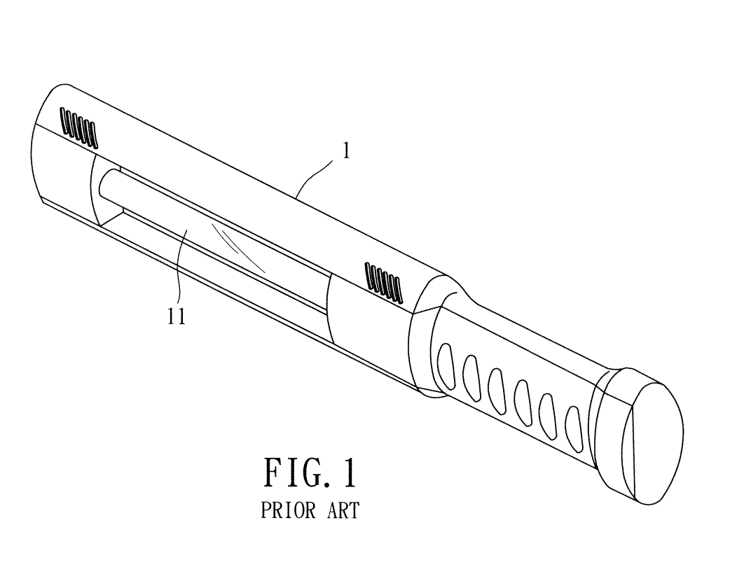

FIG. 2 is an assembled perspective view of the preferred embodiment of a bactericidal lighting device according to the present invention;

FIG. 3 is an exploded perspective view of the preferred embodiment of a bactericidal lighting device according to the present invention; and

FIG. 4 is a schematic cross-sectional side view of the preferred embodiment of a bactericidal lighting device according to the present invention.

### DETAILED DESCRIPTION OF THE PREFERRED EMBODIMENT

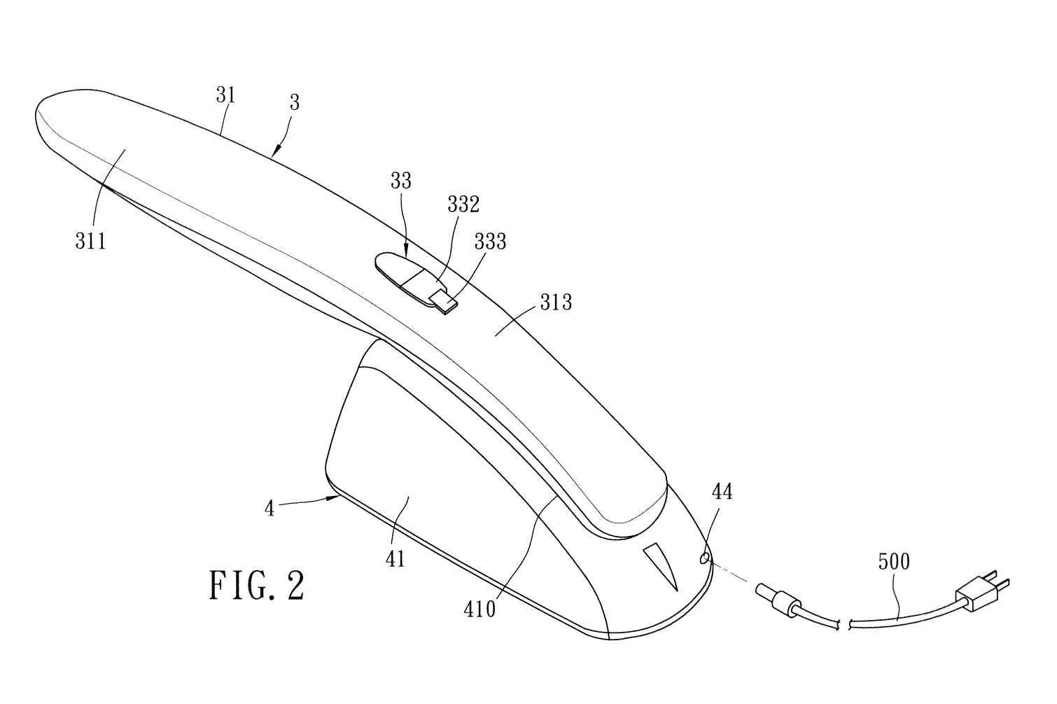

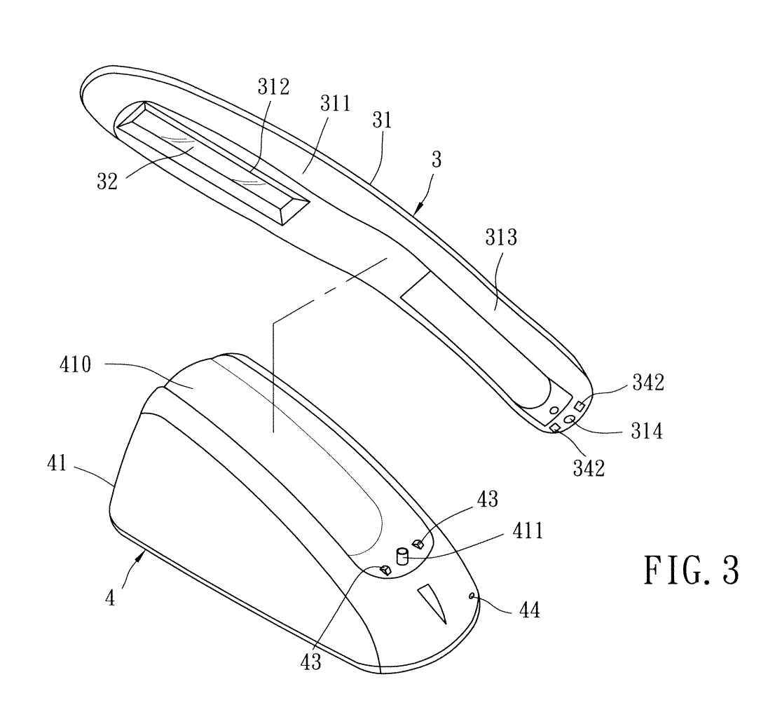

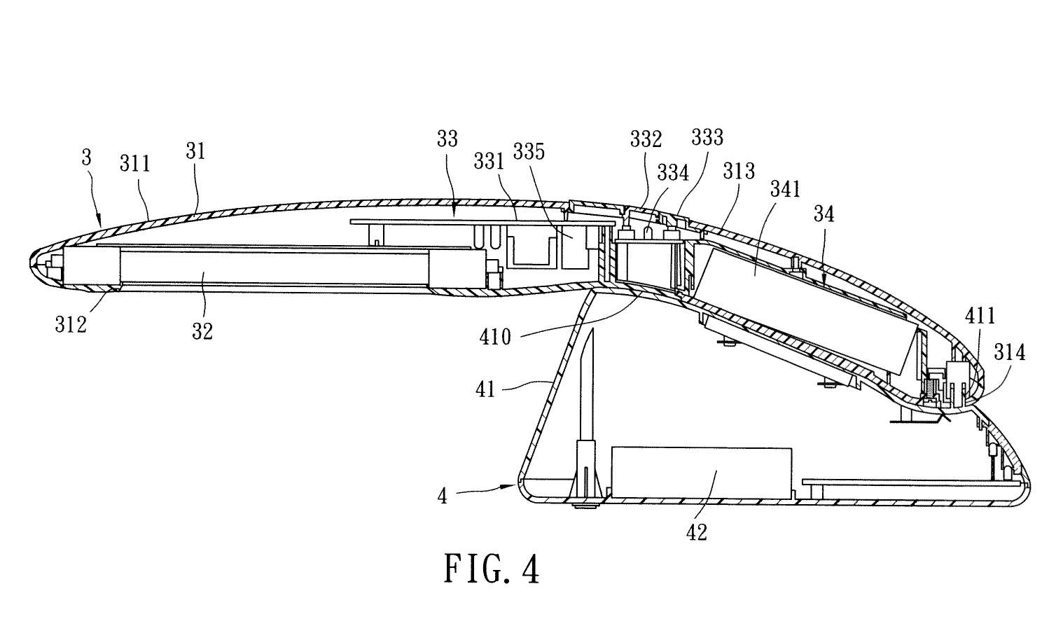

Referring to FIGS. 2 to 4, the preferred embodiment of a bactericidal lighting device according to the present invention is shown to include a bactericidal lighting bar 3 for generating ultraviolet light, and a charging base 4.

The bactericidal lighting bar 3 includes a housing 31 having one side formed with an opening 312, and a bactericidal lamp unit 32, a control unit 33 and a power supply 34 mounted in the housing 31.

In this embodiment, the housing 31 is elongated and includes a lamp-containing section 311 formed with the opening 312, a grip section 313 for gripping. The bactericidal lamp unit 32 is disposed in the lamp-containing section 311. Preferably, the lamp-containing section 311 bends from the grip section 313 to form an angle therewith.

The bactericidal lamp unit 32 is operable to generate ultraviolet light that exits the housing 31 via the opening 312. Preferably, the bactericidal lamp unit 32 uses a UV-C lamp tube.

The control unit 33 is connected electrically to the bactericidal lamp unit 32, and includes a control circuit 331, an activate key 332, a safety switch 335, and an override key 333. The control circuit 331 is disposed in the housing 31, and is operable to activate and deactivate the bactericidal lamp unit 32. The activate key 332 is accessible from an exterior of the housing 31, is coupled to the control circuit 331, and is operable to control activation of the control circuit 331 for activating the bacterial lamp unit 32. In this embodiment, the activate key 332 is disposed on a top side of the grip section 313 of the housing 31.

The safety switch 335 is disposed in the housing 31, is coupled to the control circuit 331, and is operable to deactivate the control circuit 331 for deactivating the bactericidal lamp unit 32 when the housing 31 is turned beyond a predetermined angle relative to a normal position. In this embodiment, the safety switch 335 is a roiling ball switch that is triggered when it is tilted beyond a predetermine dangle, so that the control circuit 331 may be deactivated for deactivating the bactericidal lamp unit 32.

The override key 333 is accessible from the exterior of the housing 31, is coupled to the control circuit 331, and is operable to selectively override the safety switch 335 such that the control circuit 331 and the bactericidal lamp unit 32 are able to remain activated when the housing 31 is turned beyond the predetermined angle relative to the normal position.

In this embodiment, the control circuit 331 operates to activate the bactericidal lamp unit 32 for a predetermined time period, such as 20 minutes, in response to control through the activate key 332.

Preferably, the control unit 33 further includes an indication lamp 334 coupled electrically to and controlled by the control circuit 331 to emit light of a first color when the bactericidal lamp unit 32 is in an activated state and to emit light of a second color for another predetermined time period, such as 20 minutes, when the bactericidal lamp unit 32 changes from the activated state to a deactivated state. In this embodiment, the indication lamp 334 is used under the activate key 332.

The power supply 34 includes a rechargeable battery 341 mounted in the housing 31 and operable to supply power to the control unit 33 and the bactericidal lamp unit 32, and a set of charging contacts 342 connected electrically to the rechargeable battery 341 and accessible from the exterior of the housing 31. In this embodiment, the charging contacts 342 are provided on a bottom side of a rear end of the grip section 313 of the housing 31.

The charging base 4 includes a base body 41 for holding removably the housing 31 of the bactericidal lighting bar 3, a power converter 42 (e.g., a transformer) disposed in the base body 41 and operable to convert input power (e.g., commercial AC power) into battery charging power, and a set of conductive terminals 43 accessible from an exterior of the base body 41, coupled to the power converter 42, and disposed to establish electrical connection with the charging contacts 342 when the housing 31 is held by the base body 41 to permit supply of the battery charging power to the rechargeable battery 341. Preferably, the base body 41 is configured to hold removably the grip section 313 of the housing 31.

In this embodiment, the base body 41 of the charging base 4 has a top side formed with a recess 410 for holding the grip section 313. The conductive terminals 43 are disposed in the recess 410.

In this embodiment, the base body 41 is provided with a power connector 44 that is adapted for connection to a power cord 500. The power converter 42 receives the input power via the power cord 500 and the power connector 44.

Preferably, one of the base body 41 and the grip section 313 is provided with a positioning protrusion 411, and the other of the base body 41 and the grip section 313 is provided with a positioning hole 314 for engaging removably the positioning protrusion 411 when the grip section 313 is held in the recess 410. In this embodiment, the positioning protrusion 411 protrudes from a rear portion of the recess 410, and the grip section 313 is provided with the positioning hole 314 at its rear end.

In this embodiment, when the activate key 332 is operated to activate the control circuit 331 for activating the bactericidal lamp unit 32 and the bactericidal lighting bar 3 is turned beyond a predetermined angle relative to a normal position, the safety switch 335 is triggered to deactivate the control circuit 331 for deactivating the bactericidal lamp unit 32 so that the bactericidal lighting bar 3 does not emit the ultraviolet light toward the face of the user. The bactericidal lighting bar 3 is operated in a 180° service range mode at this time. On the other hand, when the override key 333 is operated to override the safety switch 335, both the control circuit 331 and the bactericidal lamp unit 32 of the bactericidal lighting bar 3 are able to remain activated without the angular restriction imposed by the safety switch 5. The bactericidal lighting bar 3 is operated in a 360° service range mode at this time.

Therefore, when the bactericidal lighting device of this invention is used to perform a bactericidal operation, the grip section 313 of the bactericidal lighting bar 3 is gripped, and one of the activate key 332 and the override key 333 is operated so as to activate the control circuit 331 for activating the bactericidal lamp unit 32. At this time, the ultraviolet light generated by the bactericidal lighting bar 3 may be directed to a desired spot for killing bacteria, and the user may be informed of the progress of the bactericidal operation through the first-color light emitted by the indication lamp 334. After the predetermined time period expires, the bactericidal lamp unit 32 is deactivated, and the indication lamp 334 emits the second-color light to indicate completion of the bactericidal operation. When the user intends to stop the bactericidal operation before the predetermined time period expires, the activate key 332 or the override key 333 may be operated once again to turn off the control circuit 331.

After using the bactericidal lighting bar 3 for some time, the power of the rechargeable battery 341 thereof may be insufficient for further operation. The power cord 500 may be connected to the power connector 44 of the charging base 4, and the grip section 313 of the bactericidal lighting bar 3 may be held removably in the recess 410, with the conductive terminals 43 establishing electrical connection with the charging contacts 342, and with the positioning protrusion 411 engaging the positioning hole 314 of the grip section 313 for positioning the bactericidal lighting bar 3 on the charging base 4. At this time, battery charging power from the charging base 4 may be used to charge the rechargeable battery 341. Moreover, the bactericidal lighting bar 3 may be used to conduct a bactericidal operation while it is held by the charging base 4. This is suitable for long-duration localized bactericidal operations since the bactericidal lighting bar 3 is not required to be held by the user.

By virtue of the designs of the bactericidal lighting bar 3 and the charging base 4, the bactericidal lighting bar 3 may be conveniently recharged, and the bactericidal lighting bar 3 may be used for long-duration localized bactericidal operations while it is held on the charging base 4. Moreover, through the design of the control unit 33 that includes the control circuit 331, the activate key 332, the safety switch 335 and the override key 333, the bactericidal lighting bar 3 is available for use in a 180° service range mode, in which the safety switch 335 imposes an angular restriction on operation of the bactericidal lighting bar 3, or a 360° service range mode, in which the angular restriction imposed by the safety switch 335 is overridden due to the override key 333.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

What is claimed is:

1. A bactericidal lighting device comprising:
a bactericidal lighting bar including
a housing having one side formed with an opening,
a bactericidal lamp unit mounted in said housing and operable to generate ultraviolet light that exits said housing via said opening,
a control unit connected electrically to said bactericidal lamp unit and including
a control circuit disposed in said housing and operable to activate and deactivate said bactericidal lamp unit,
an activate key accessible from an exterior of said housing, coupled to said control circuit, and operable to control activation of said control circuit for activating said bactericidal lamp unit,
a safety switch coupled to said control circuit and operable to deactivate said control circuit for deactivating said bactericidal lamp unit when said housing is turned beyond a predetermined angle relative to a normal position, and
an override key accessible from the exterior said housing, coupled to said control circuit, and operable to selectively override said safety switch such that said control circuit and said bactericidal lamp unit are able to remain activated when said housing is turned beyond the predetermined angle relative to the normal position, and
a power supply including
a rechargeable battery mounted in said housing and operable to supply power to said control unit and said bactericidal lamp unit, and
a set of charging contacts connected electrically to said rechargeable battery and accessible from the exterior of said housing; and
a charging base including
a base body for holding removably said housing of said bactericidal lighting bar,
a power converter disposed in said base body and operable to convert input power into battery charging power, and
a set of conductive terminals accessible from an exterior of said base body, coupled to said power converter, and disposed to establish electrical connection with said charging contacts when said housing is held by said base body to permit supply of the battery charging power to said rechargeable battery.

2. The bactericidal lighting device as claimed in claim 1, wherein said control unit operates to activate said bactericidal lamp unit for a predetermined time period in response to control through said activate key.

3. The bactericidal lighting device as claimed in claim 2, wherein said control unit further includes an indication lamp coupled electrically to and controlled by said control circuit to emit light of a first color when said bactericidal lamp unit is in an activated state and to emit light of a second color when said bactericidal lamp unit changes from the activated state to a deactivated state.

4. The bactericidal lighting device as claimed in claim 1, wherein said housing is elongated and includes a lamp-containing section formed with said opening, and a grip section for gripping, said bactericidal lamp unit being disposed in said lamp-containing section, said grip section being held removably by said base body.

5. The bactericidal lighting device as claimed in claim 4, wherein said lamp-containing section bends from said grip section to form an angle therewith.

6. The bactericidal lighting device as claimed in claim 4, wherein said base body has a top side formed with a recess for holding said grip section, said conductive terminals being disposed in said recess.

7. The bactericidal lighting device as claimed in claim 6, wherein one of said base body and said grip section is provided with a positioning protrusion, and the other of said base body and said grip section is provided with a positioning hole for engaging removably said positioning protrusion when said grip section is held in said recess.
